(19) United States
(12) Patent Application Publication
(10) Pub. No.: US 2025/0115859 A1
(43) Pub. Date: Apr. 10, 2025

(54) CELL PROCESSING SYSTEM

(71) Applicants: Canon Medical Systems Corporation, Otawara-shi (JP); CANON KABUSHIKI KAISHA, Tokyo (JP)

(72) Inventors: Yosuke YAMAGUCHI, Kawasaki (JP); Yasuo SAKURAI (DECEASED); Tetsuya SAKAI, Ryugasaki (JP); Tomoki HANEISHI, Tokyo (JP); Takaaki KAWANO, Chigasaki (JP); Hiroshi SUZUKI, Tokyo (JP); Tomoaki YAMAKI, Yokohama (JP)

(73) Assignees: Canon Medical Systems Corporation, Otawara-shi (JP); CANON KABUSHIKI KAISHA, Tokyo (JP)

(21) Appl. No.: 18/907,103

(22) Filed: Oct. 4, 2024

(30) Foreign Application Priority Data
Oct. 6, 2023 (JP) 2023-174686

(51) Int. Cl. C12M 1/34; C12M 1/26; C12M 1/36

(52) U.S. Cl. CPC C12M 41/36; C12M 33/00; C12M 41/32; C12M 41/44; C12M 41/48

(57) ABSTRACT

A cell processing system according to the embodiment includes an introducing device, a culturing device, and an assessing apparatus. The introducing device introduces reprogramming factors into target cells derived from a sample of a donor to develop iPS cells from the target cells. The culturing device cultures the iPS cells developed from the target cells in a culture vessel. The assessing apparatus individually assesses at least an input to and/or an output from the introducing device and an input to and/or an output from the culturing device.

### CROSS-REFERENCE TO RELATED APPLICATION

This application is based upon and claims the benefit of priority from Japanese Patent Application No. 2023-174686, filed Oct. 6, 2023, the entire contents of which are incorporated herein by reference.

### FIELD

Embodiments described herein relate generally to a cell processing system.

### BACKGROUND

Induced pluripotent stem (iPS) cells are generated manually. In order to generate iPS cells at a low cost with a high quality, there is a demand for automated control of the iPS cell generating process. To realize automated control, the conditions for each processing step included in the generating process need to be optimized. However, the conditions for each processing step are affected by variations in operation procedures and outputs due to the processing steps being performed manually.

### BRIEF DESCRIPTION OF THE DRAWINGS

FIG. 1 is a diagram showing a configuration example of a cell processing system according to the present embodiment.

FIG. 2 is a diagram showing a configuration example of a flow path between an iPS cell generating system and a measuring apparatus shown in FIG. 1.

FIG. 3 is a diagram showing a configuration example of an assessing apparatus.

FIG. 4 is a diagram showing a procedure for an optimization process of processing conditions for an optimization target unit by the cell processing system.

FIG. 5 shows an example of a display screen for results of assessment.

FIG. 6 schematically shows optimization of a processing condition for a first optimization target unit (factor introducing device) based on a result of assessment of a first output and optimization of a processing condition for a second optimization target unit (iPS cell culturing device) based on a result of assessment of a second output.

FIG. 7 is a diagram schematically showing optimization of a processing condition for each independent processing step.

FIG. 8 is a diagram schematically showing optimization of processing conditions for partially linked steps.

FIG. 9 is a diagram schematically showing optimization of a processing condition for a fully linked step.

### DETAILED DESCRIPTION

A cell processing system according to the embodiment includes an introducing unit, a culturing unit, and an assessing apparatus. The introducing unit introduces one or more reprogramming factors into one or more target cells derived from a sample of a donor to develop one or more iPS cells from the target cells. The culturing unit cultures the iPS cells developed from the target cells in a culture vessel. The assessing apparatus individually assesses at least an input to and/or an output from the introducing unit and an input to and/or an output from the culturing unit.

Hereinafter, the cell processing system according to the present embodiment will be described in detail with reference to the accompanying drawings.

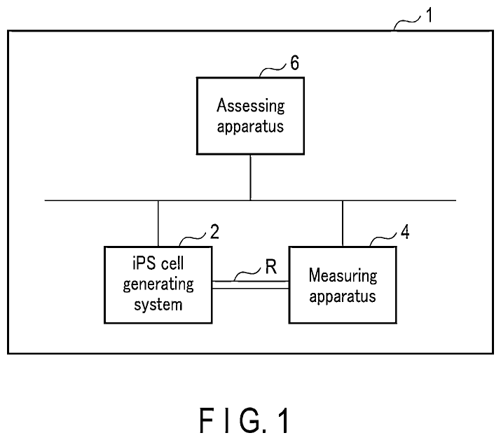

FIG. 1 is a diagram showing a configuration example of a cell processing system 1 according to the present embodiment. As shown in FIG. 1, the cell processing system 1 includes an iPS cell generating system 2, a measuring apparatus 4, and an assessing apparatus 6. The iPS cell generating system 2 and the measuring apparatus 4 are connected via a flow path R through which various cell fluids, etc. flow. The iPS cell generating system 2, the measuring apparatus 4, and the assessing apparatus 6 may be mutually connected via wire or wirelessly so as to enable information communication thereamong.

The iPS cell generating system 2 is a mechanical system that generates, from a sample of a donor, induced pluripotent stem (iPS) cells derived from the donor. The measuring apparatus 4 is a mechanical device adapted to measure, for one or more assessment items, an input to and/or an output from each processing step of the generating process by the iPS cell generating system 2. The assessing apparatus 6 is a computer that assesses results of measurement of the input and/or the output for the assessment items.

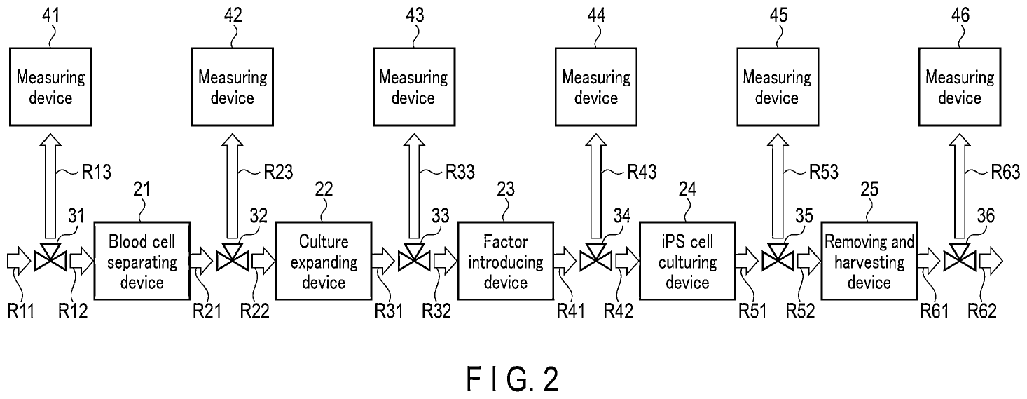

FIG. 2 is a diagram showing a configuration example of a flow path between the iPS cell generating system 2 and the measuring apparatus 4 shown in FIG. 1. As shown in FIG. 2, the iPS cell generating system 2 includes a blood cell separating device 21, a culture expanding device 22, a factor introducing device 23, an iPS cell culturing device 24, and a removing and harvesting device 25. It is assumed that each of the blood cell separating device 21, the culture expanding device 22, the factor introducing device 23, the iPS cell culturing device 24, and the removing and harvesting device 25 is automatically controlled.

The blood cell separating device 21 is a mechanical device adapted to separate target cells by removing unnecessary substances from the sample of the donor. It is assumed that the sample is blood of the donor. The target cells are not particularly limited to specific cells, and may be any cells that can be transformed into iPS cells; however, in the present embodiment, it is assumed that the target cells are CD34-positive cells (CD34+ cells) contained in peripheral blood mononuclear cells (PBMCs). The blood cell separating device 21 is adapted to remove plasma and red blood cells from the blood to extract PBMCs containing CD34-positive cells. Blood is input to the blood cell separating device 21, and CD34-positive cells or PBMCs are output from the blood cell separating device 21. Examples of the blood cell separating device 21 include a filter and a helical flow path. The processing steps of the blood cell separating device 21 are automated, and are conducted in accordance with blood cell separation conditions, which are processing conditions set in advance. The blood cell separation conditions may include, in the case of using a filter, the type and coarseness of the filter, the fluid delivery pressure, etc. The blood cell separating conditions may include, in the case of using a helical flow path, the shape of the flow path, the fluid delivery pressure, etc. The blood cell separation conditions may further include the type of the blood cell separating device 21, for example, which of a filter, a helical flow path, etc. is to be used.

The culture expanding device 22 is a mechanical device adapted to culture-expand PBMCs. Through the culture expansion of the PBMCs, CD34-positive cells present in the PBMCs expand together with the other cells. As an example, the culture expanding device 22 includes a culture vessel, a dispenser, and a controller. The culture vessel is a vessel in which PBMCs are cultured. The dispensing unit is a mechanical device including a cylinder and a pump adapted to dispense PBMCs to the culture vessel. The controller is a mechanical device adapted to control a culture environment in the culture vessel. PBMCs are input to the culture expanding device 22, and culture-expanded PBMCs are output from the culture expanding device 22. The processing steps of the culture expanding device 22 are automated, and are conducted in accordance with culture expanding conditions, which are processing conditions set in advance. The culture expanding conditions may include a temperature, a humidity, a carbon dioxide concentration, a culture expansion time, etc.

The factor introducing device 23 is a mechanical device adapted to introduce reprogramming factors into target cells derived from a sample of a donor to develop iPS cells from the target cells. Specifically, the factor introducing device 23 introduces reprogramming factors into CD34-positive cells present in PBMCs that have been culture-expanded by the culture expanding device 22. After the introduction of the reprogramming factors into the CD34-positive cells, the CD34-positive cells are reprogrammed, and iPS cells are developed. The factor introducing device 23 includes, for example, a culture vessel, a fluid delivery device, and a controller. The culture vessel is a container for holding CD34-positive cells and iPS cells. The fluid delivery unit is a mechanical device including a cylinder and a pump for delivering various cell fluids and reagents. Specifically, the fluid delivery unit delivers cell fluids containing CD34-positive cells to the culture vessel, and supplies a reagent fluid containing reprogramming factors to the culture vessel in which the cell fluids containing the CD34-positive cells are accumulated. The reprogramming factors, also known as a Yamanaka factors, are adapted to reprogram target cells such as CD34-positive cells. Specifically, the reprogramming factors may be Oct family genes, Klf family genes, or Myc family genes, or gene products thereof. Examples of the Oct family genes include Oct3/4, examples of the Klf family genes include Klf4, and examples of the Myc family genes include c-Myc and L-Myc. The reprogramming factors may be Sox family genes or gene products thereof. Examples of the Sox family genes include Sox2. The controller is a mechanical device adapted to control a culture environment in the culture vessel. Target cells such as CD34-positive cells are input to the factor introducing device 23, and iPS cells are output from the factor introducing device 23. The processing steps of the factor introducing device 23 are automated, and are conducted in accordance with developing conditions, which are processing conditions set in advance. The developing conditions may include a temperature, a humidity, and a carbon dioxide concentration, etc. in the developing step, as well as the amount of reprogramming factors.

The iPS cell culturing device 24 is a mechanical device adapted to culture iPS cells developed from target cells by the factor introducing device 23. Specifically, the iPS cell culturing device 24 includes a culture vessel, a fluid delivery device, and a controller. The culture vessel is a vessel for culturing iPS cells. The fluid delivery unit is a mechanical device including a cylinder and a pump for dispensing cell fluids containing iPS cells to the culture vessel. The fluid delivery unit suitably adds various types of reagents such as media to the culture vessel. Over a culture period, multiple cell lumps (colonies) of iPS cells are formed in the culture vessel. Developed iPS cells are input to the iPS cell culturing device 24, and cultured iPS cells are output from the iPS cell culturing device 24. The processing steps of the iPS cell culturing device 24 are automated, and are conducted in accordance with culturing conditions, which are processing conditions set in advance. The culturing conditions may include a temperature, a humidity, a carbon dioxide concentration, components of media, a frequency of supplying media, a culture time, etc.

The removing and harvesting device 25 is a mechanical device adapted to remove iPS cells cultured in the iPS cell culturing device 24 from the culture vessel of the iPS cell culturing device 24 and harvest them. Specifically, the removing and harvesting device 25 includes a removing unit and a harvesting device. The removing unit is a mechanical device adapted to remove iPS cells from the culture vessel. The removal method is not limited to a particular one. Examples include a method of mechanically removing iPS cells by scrapers, etc., a method of spraying a given fluid such as media and phosphate buffered saline (PBS) onto the iPS cells and removing them, and a method of removing the iPS cells by a chemical reaction with various types of enzymes and reagents. The harvesting device is a mechanical device including a cylinder and a pump for aspirating cell lumps of iPS cells removed from the culture vessel and delivering them to a given container. Cultured iPS cells are input to the removing and harvesting device 25, and iPS cells removed by the removing unit and/or iPS cells harvested by the harvesting device are output from the removing and harvesting device 25. The processing steps of the removing and harvesting device 25 are automated, and are conducted in accordance with removing and harvesting conditions, which are processing conditions set in advance. The removing and harvesting conditions may include a type of removal method, an intensity and a number of removal operations, etc. Note that the removing and harvesting device 25 may be integrally formed with the iPS cell culturing device 24.

As shown in FIG. 2, the measuring apparatus 4 includes measuring devices 41 to 46. The measuring device 41 is a mechanical device adapted to measure an input to the blood cell separating device 21 for one or more assessment items. Examples of an assessment item include the number of cells present in the blood. The types of the cells may be platelets, white blood cells, red blood cells, and the like. The number of cells can be measured by a flow cytometer, a cell counter, or the like. The flow cytometer and the cell counter are examples of the measuring device 41. As an assessment item, a presence or absence of an infectious disease in the donor may be tested. The presence or absence of an infectious disease in the donor can be tested by various inspection kits. Various inspection kits are examples of the measuring device 41.

The measuring device 42 is a mechanical device adapted to measure an output from the blood cell separating device 21 and/or an input to the culture expanding device 22 for one or more assessment items. Examples of the assessment item for the output from the blood cell separating device 21 include a total number of cells and a ratio of living cells present in the output. Examples of the assessment items for the input to the culture expanding device 22 include a ratio of CD34-positive cells present in the input. As the measuring device 42 for measurement for the assessment items, a flow cytometer, a cell counter, a cell sorter, etc., may be employed. The flow cytometer, the cell counter, and the cell sorter are examples of the measuring device 42.

The measuring device 43 is a mechanical device adapted to measure an output from the culture expanding device 22 and/or an input to the factor introducing device 23 for one or more assessment items. Examples of the assessment items include a ratio of CD34-positive cells, a total number of cells, and a ratio of living cells present in the output and/or the input. Measurement for such assessment items can be performed by a flow cytometer, a cell counter, a cell sorter, etc. The flow cytometer, the cell counter, and the cell sorter are examples of the measuring device 43.

The measuring device 44 is a mechanical device adapted to measure an output from the factor introducing device 23 and/or an input to the iPS cell culturing device 24 for one or more assessment items. Examples of the assessment items include a total number of cells and a ratio of living cells present in the output and/or the input. Measurement for such assessment items can be performed by a flow cytometer, a cell counter, a cell sorter, etc. The flow cytometer, the cell counter, and the cell sorter are examples of the measuring device 44.

The measuring device 45 is a mechanical device adapted to measure an output from the iPS cell culturing device 24 and/or an input to the removing and harvesting device 25 for one or more assessment items. Examples of the assessment items include a number, a size, and an intra-vessel unevenness of cell lumps present in the output and/or input. Measurement for such assessment items can be performed by macroscopic observation by a measurer using a microscope or image processing of a microscopic image. A microscope and an image processor are examples of the measuring device 45.

The measuring device 46 is adapted to measure an output from the removing and harvesting device 25 for one or more assessment items. Examples of the assessment items include a number, an undifferentiated cell marker positive rate, and a reprogramming factor retention rate of cells present in the output. Measurement for such assessment items can be performed by a flow cytometer, a cell counter, a cell sorter, etc. The flow cytometer, the cell counter, and the cell sorter are examples of the measuring device 46.

As shown in FIG. 2, the blood cell separating device 21, the culture expanding device 22, the factor introducing device 23, the iPS cell culturing device 24, and the removing and harvesting device 25 are connected in order via flow paths R.

An inlet of a flow path R11 is connected to an outlet of a container for accumulating a donor sample, which is an input to the blood cell separating device 21, and the input flows into the blood cell separating device 21 via the flow path R11 and a flow path R12. The flow paths R11 and R12, through which the input to the blood cell separating device 21 flows, are connected to the blood cell separating device 21. Flow paths R21 and R22, through which an output from the blood cell separating device 21, namely, an input to the culture expanding device 22, flows, are connected between the blood cell separating device 21 and the culture expanding device 22. Flow paths R31 and R32, through which an output from the culture expanding device 22, namely, an input to the factor introducing device 23, flows, are connected between the culture expanding device 22 and the factor introducing device 23. Flow paths R41 and R42, through which an output from the factor introducing device 23, namely, an input to the iPS cell culturing device 24, flows, are connected between the factor introducing device 23 and the iPS cell culturing device 24. Flow paths R51 and R52, through which an output from the iPS cell culturing device 24, namely, an input to the removing and harvesting device 25, flows, is connected between the iPS cell culturing device 24 and the removing and harvesting device 25. Flow paths R61 and R62, through which an output from the removing and harvesting device 25 flows, is connected to the removing and harvesting device 25. A container for accumulating an output from the removing and harvesting device 25 is connected to an outlet of the flow path R62.

It is expected that an iPS cell culture vessel passes through the flow paths R51, R52, and R53. In this case, a belt conveyor or various types of carrying devices using a rack and pinion, a ball screw, etc. may be used as the flow paths R51, R52, and R53. Vinyl tubes, resin tubes, metal tubes, etc. may be used as other flow paths R, through which various cell fluids are expected to flow. If the removing and harvesting device 25 is integrally formed with the iPS cell culturing device 24, the flow paths R51, R52, and R53 need not be provided. If the flow path R53 is not provided, the microscope provided in the measuring device 45 is arranged so as to face the culture surface of the culture vessel placed in the iPS cell culturing device 24, thus allowing for measurement of iPS cells being cultured.

The measuring device 41 is connected to a flow path R13, which branches off from the flow paths R11 and R12 via a switch 31. The switch 31 adjusts an amount of flow of the donor sample (blood) to the flow path R13 and an amount of flow of the donor sample (blood) to the flow path R12. The measuring device 42 is connected to a flow path R23, which branches off from the flow paths R21 and R22 via a switch 32. The switch 32 adjusts a distribution of the output from the blood cell separating device 21 between an amount of flow to the flow path R23 and an amount of flow to the flow path R22. The measuring device 43 is connected to a flow path R33, which branches off from the flow paths R31 and R32 via a switch 33. The switch 33 adjusts a distribution of the output from the culture expanding device 22 between an amount of flow to the flow path R33 and an amount of flow to the flow path R32. The measuring device 44 is connected to a flow path R43, which branches off from the flow paths R41 and R42 via a switch 34. The switch 34 adjusts a distribution of the output from the factor introducing device 23 between the amount of flow to the flow path R43 and the amount of flow to the flow path R42. The measuring device 45 is connected to a flow path R53, which branches off from the flow paths R51 and R52 via a switch 35. The switch 35 adjusts a distribution of an amount of flow of the output from the iPS cell culturing device 24 between an amount of flow to the flow path R53 and an amount of flow to the flow path R52. The measuring device 46 is connected to a flow path R63, which branches off from the flow paths R61 and R62 via a switch 36. The switch 36 adjusts a distribution of an amount of flow of the output from the removing and harvesting device 25 between an amount of flow to the flow path R63 and an amount of flow to the flow path R62.

Herein, the switches 31, 32, 33, 34, 35, and 36 will be collectively referred to as a “switch 3q”, the flow paths R12, R22, R32, R42, R52, and R62, which are the main flow paths, will be collectively referred to as a “main flow path Rr2”, and the flow paths R13, R23, R33, R43, R53, and R63, which are branching flow paths, will be collectively referred to as a “branching flow path Rr3”. It is assumed that “q” is an index that takes a natural number from 1 to 6 for distinguishing between the switches, and that “r” is an index that takes a natural number from 1 to 6 for distinguishing between the flow paths. There is no restriction to the distribution between the amount of flow to the main flow path Rr2 and the amount of flow to the branching flow path Rr3. For example, a distribution to only one of the main flow path Rr2 and the branching flow path Rr3 may be adopted, an even distribution to both the main flow path Rr2 and to the branching flow path Rr3 may be adopted, or an uneven (e.g., 9:1) distribution may be adopted. The switch 3q may be of a manually operable type, or of a type that is electromagnetically, mechanically, or electrically operable without manual intervention.

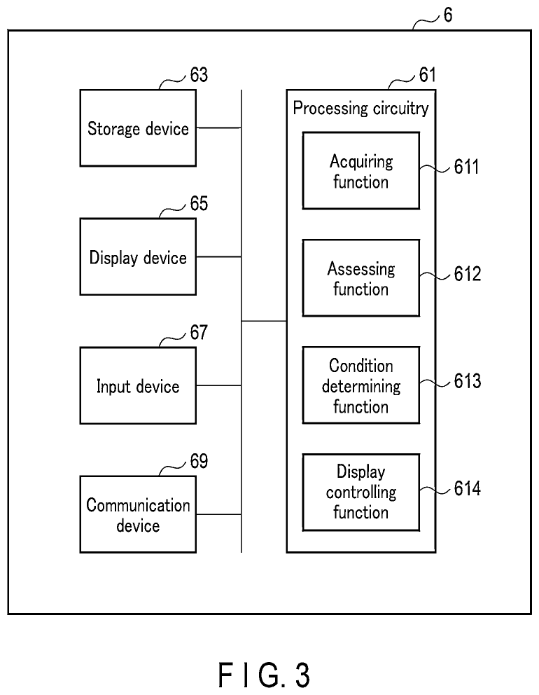

FIG. 3 is a diagram showing a configuration example of the assessing apparatus 6. As shown in FIG. 3, the assessing apparatus 6 includes processing circuitry 61, a storage device 63, a display device 65, an input device 67, and a communication device 69. The processing circuitry 61, the storage device 63, the display device 65, the input device 67, and the communication device 69 are mutually connected via a bus so as to enable communication thereamong.

The processing circuitry 61 includes a processor. The processor executes programs related to the present embodiment, and realizes at least one of an acquiring function 611, an assessing function 612, a condition determining function 613, and a display controlling function 614. Such programs are stored in the storage device 63 or a computer-readable storage medium such as a portable recording medium.

By realizing the acquiring function 611, the processing circuitry 61 acquires a variety of information. For example, the processing circuitry 61 acquires, from the measuring apparatus 4, results of measurement of an input to and/or an output from an optimization target unit 2n for various assessment items. Herein, the optimization target unit 2n denotes a unit on which optimization of processing conditions, which are operation conditions for a processing step to be performed by the unit, is performed. Specifically, the optimization target unit 2n denotes some or all of the blood cell separating device 21, the culture expanding device 22, the factor introducing device 23, the iPS cell culturing device 24, and the removing and harvesting device 25. Note that “n” is an index that takes a natural number from 1 to 5 for denoting a specific optimization target unit.

By realizing the assessing function 612, the processing circuitry 61 assesses an input to and/or an output from the optimization target unit 2n. More specifically, with the acquiring function 611, the processing circuitry 61 assesses an input to and/or an output from the optimization target unit 2n, based on the results of measurement for assessment items acquired from the optimization target unit 2n. Herein, the processing circuitry 61 individually assesses at least an input to and/or an output from the factor introducing device 23 and an input to and/or an output from the iPS cell culturing device 24.

By realizing the condition determining function 613, the processing circuitry 61 determines processing conditions for the optimization target unit 2n in accordance with results of assessment by the assessing function 612. The processing circuitry 61 may assign the determined processing conditions to the optimization target unit 2n.

By realizing the display controlling function 614, the processing circuitry 61 displays a variety of information on the display device 65. For example, the processing circuitry 61 displays the results of measurement by the measuring apparatus 4, the results of assessment by the assessing function 612, the processing conditions for the optimization target unit 2n, etc.

The storage device 63 is a storage device that stores various types of information, such as a RAM, a ROM, a hard disk drive (HDD), a solid-state drive (SSD), a semiconductor storage device, etc. The storage device stores, for example, various types of programs, etc. The storage device 63 implemented as hardware may be a drive assembly configured to read and write various types of information from and into portable storage media such as a CD-ROM drive, a DVD drive, a flash memory, etc.

The display device 65 displays a variety of information in accordance with the display controlling function 614. For the display device 65, a cathode-ray tube (CRT) display, a liquid crystal display, an organic EL display, an LED display, a plasma display, or any other displays known in the present technical field may be suitably employed. The display device 65 may be a projector.

The input device 67 is an interface via which various instructions from the operator are input. For the input device 67, a keyboard, a mouse, various types of switches, etc. may be employed. In response to various instructions, the input device 67 supplies output signals to the processing circuitry 61 via a bus.

The communication device 69 includes a network interface card (NIC) for performing data communications with external devices such as various devices in the cell processing system 1, a workstation, a picture archiving and communication system (PACS), a radiological information system (RIS), and a hospital information system (HIS).

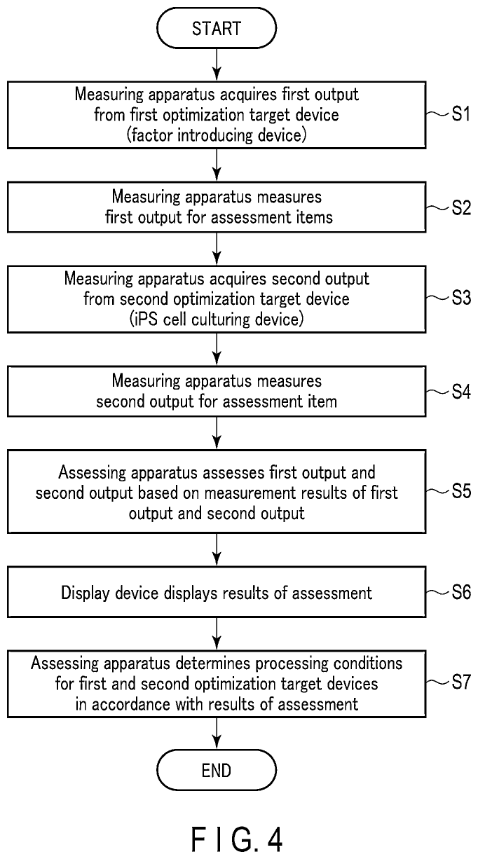

Next, an example of an optimization process of processing conditions for an optimization target unit 2n by the cell processing system 1 will be described. FIG. 4 is a diagram showing a procedure for an optimization process of processing conditions for the optimization target unit 2n by the cell processing system 1. It is assumed that, in the embodiment shown in FIG. 4, a sample is human blood, and that target cells are CD34-positive cells.

It is also assumed that the optimization process in the embodiment shown in FIG. 4 is conducted in parallel with an iPS cell generating process by the iPS cell generating system 2. In this case, for the iPS cell generating process, the switches 3q are opened by a predetermined amount on the side of the main flow paths Rr2 to allow various cell fluids to flow through the blood cell separating device 21, the culture expanding device 22, the factor introducing device 23, the iPS cell culturing device 24, and the removing and harvesting device 25; furthermore, for measurement of an input to and an output from the optimization target unit 2n, a corresponding switch 3q is opened by a predetermined amount on the side of the branching flow path Rr3. The other switches 3q are closed on the side of the branching flow path Rr3. The optimization target unit 2n may be set in some or all of the blood cell separating device 21, the culture expanding device 22, the factor introducing device 23, the iPS cell culturing device 24, and the removing and harvesting device 25.

For concreteness, it is assumed that the optimization target units 2n are the factor introducing device 23 and the iPS cell culturing device 24. In this case, the switch 31 is operated so that the branching flow path R13 is closed and the main flow path R12 is open, the switch 32 is operated so that the branching flow path R23 is closed and the main flow path R22 is open, the switch 33 is operated so that the branching flow path R33 is closed and the main flow path R32 is open, the switch 34 is operated so that both of the branching flow path R43 and the main flow path R42 are open, the switch 35 is operated so that both of the branching flow path R53 and the main flow path R52 are open, and the switch 36 is operated so that the branching flow path R63 is closed and the main flow path R62 is open. Accordingly, the donor blood flows from a container in which the donor blood is accumulated into the blood cell separating device 21 via the flow paths R11 and R12, PBMCs output from the blood cell separating device 21 flow into the culture expanding device 22 via the flow paths R21 and R22, PBMCs output from the culture expanding device 22 flow into the factor introducing device 23 via the flow paths R31 and R32, a fluid (hereinafter, an “iPS cell fluid”) containing iPS cells output from the factor introducing device 23 flows into the iPS cell culturing device 24 via the flow paths R41 and R42, and flows into the measuring device 44 via the flow path R43, a culture vessel holding iPS cells output from the iPS cell culturing device 24 is conveyed to the removing and harvesting device 25 via the flow paths R51 and R52 and is conveyed to the measuring device 45 via the flow path R53, and the iPS cell fluid output from the removing and harvesting device 25 flows into a container via the flow paths R61 and R62.

As shown in FIG. 4, the measuring apparatus 4 acquires a first output from a first optimization target unit (factor introducing device 23) (step S1). The measuring device 43 acquires, as the first output, a suspension (iPS cell fluid) containing iPS cells developed by the factor introducing device 23.

After step S1, the measuring apparatus 4 measures the first output acquired at step S1 for one or more assessment items (step S2). The measuring device 43 performs, at step S2, measurement for the assessment items such as a total number of cells (iPS cell number) and/or a ratio of living cells present in the iPS cell fluid. The types of assessment items for the measurement target can be freely selected. The data of the results of measurement for the assessment items is transferred to the assessing apparatus 6.

After step S2, the measuring apparatus 4 acquires a second output form a second optimization target unit (iPS cell culturing device 24) (step S3). The measuring device 45 acquires, as the second output, iPS cells cultured by the iPS cell culturing device 24. More specifically, the measuring device 45 acquires a culture vessel in which cell lumps of the cultured iPS cells are formed.

After step S3, the measuring apparatus 4 measures the second output acquired at step S3 for one or more assessment items (step S4). At step S4, the measuring device 44 performs measurement for the assessment items such as a number, a size, and/or an intra-vessel unevenness of cell lumps (colonies) of the acquired iPS cells. The types of assessment items for the measurement target can be freely selected. The data of the results of measurement for the assessment items is transferred to the assessing apparatus 6.

After step S4, the assessing apparatus 6 assesses the first output and the second output based on results of measurement of the first output obtained at step S2 and results of measurement of the second output obtained at step S4 (step S5). At step S5, the processing circuitry 61 of the assessing apparatus 6 outputs, with the assessing function 612, comparisons of the results of measurement of the first and second outputs against separately defined standards as results of assessment. As an example, the processing circuitry 61 outputs results of determination as to whether each of the total number of cells and/or the ratio of living cells present in the iPS cell fluid, which are the results of measurement of the first output from the factor introducing device 23, satisfies standards. The results of determination are output as results of assessment. As another example, the processing circuitry 61 may output, as the results of assessment, a degree to which each of the results of measurement exceeds or falls below the standards.

Similarly, the processing circuitry 61 outputs results of assessment of the number, the size, and/or the intra-vessel unevenness of cell lumps, which are the results of measurement of the second output from the iPS cell culturing device 24.

After step S5, the processing circuitry 61 displays, with the display controlling function 614, the results of assessment output at step S5 on the display device 65 (step S6). The processing circuitry 61 displays the results of assessment of the first output and the results of assessment of the second output.

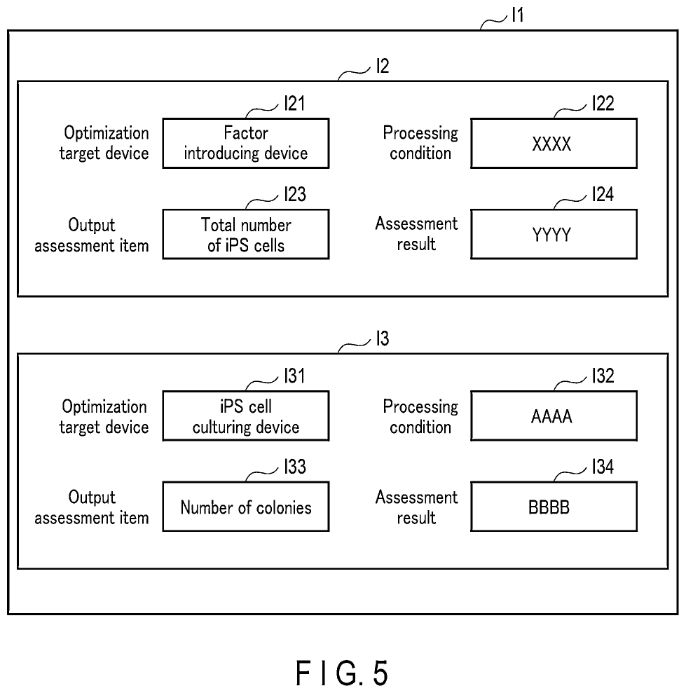

FIG. 5 shows an example of a display screen I1 for the results of assessment. As shown in FIG. 5, a display section I2 for the results of assessment of the first output from the factor introducing device 23 and a display section I3 for the results of assessment of the second output from the iPS cell culturing device 24 are provided in the display screen I1.

As shown in FIG. 5, display sections I21, I22, I23, and I24 are provided in the display section I2. In the display section I21, the name of the first optimization target unit, such as “factor introducing device”, is displayed. In the display section I22, a condition value of the current processing condition for the first optimization target unit, such as “XXXX”, is displayed. If there are multiple processing conditions, all the conditions may be displayed, or only one or more conditions to be optimized may be displayed. In the display section I23, the name of an item to be assessed for the first output from the first optimization target unit, such as “total number of iPS cells”, is displayed. In the display section I24, a result of assessment of the first output for the item to be assessed, such as “YYYY”, is displayed. If there are multiple items to be assessed, the display sections I23 and I24 can be arranged in the same number as the number of the items to be assessed.

As shown in FIG. 5, display sections I31, I32, I33, and I34 are provided in the display section I3. In the display section I31, the name of the second optimization target unit, such as “iPS cell culturing device”, is displayed. In the display section I32, a condition value of the current processing condition for the second optimization target unit, such as “AAAA”, is displayed. If there are multiple processing conditions, all the conditions may be displayed, or only one or more conditions to be optimized may be displayed. In the display section I33, the name of an item to be assessed for the second output from the second optimization target unit, such as “number of colonies”, is displayed. In the display section I34, a result of assessment of the second output for the item to be assessed, such as “BBBB”, is displayed. If there are multiple items to be assessed, the display sections I33 and I34 can be arranged in the same number as the number of the items to be assessed.

Through the displaying of the results of assessment of the first output and the second output as shown in FIG. 5, the operator can determine whether or not the processing conditions for each of the first optimization target unit (the factor introducing device 23) and the second optimization target unit (the iPS cell culturing device 24) are appropriate, and can verify the technical feasibility of each of the steps to be automated in the first optimization target unit (the factor introducing device 23) and the second optimization target unit (the iPS cell culturing device 24).

After step S6, the assessing apparatus 6 determines, with the condition determining function 613, processing conditions for the first and second optimization target units (the factor introducing device 23 and the iPS cell culturing device 24) in accordance with the results of assessment obtained at step S5 (step S7). At step S7, the processing circuitry 61 of the assessing apparatus 6 determines, with the condition determining function 613, a condition value of a processing condition for the factor introducing device 23 in accordance with the results of assessment of an input to and/or an output from the factor introducing device 23. As an example, if the results of assessment show that the results of measurement of the input to and/or the output from the factor introducing device 23 satisfy standards, the condition value of the processing condition related to the input and/or output is maintained. As another example, if the results of assessment show that the results of measurement of the input to and/or the output from the factor introducing device 23 do not satisfy the standards, the condition value of the processing condition related to the input and/or output is updated. It can be expected that the updated condition value is to be input via the input device 67 by an operator who has confirmed the results of assessment at step S6. As another example, the processing circuitry 61 may determine, as the updated condition value, a value increased or decreased by a predetermined value from the currently set value, or a random value. As another example, the processing circuitry 61 may determine, as an updated upper-limit value, a value increased or decreased by a value corresponding to a degree to which each of the results of measurement exceeds or falls below the standards.

At step S7, the processing circuitry 61 may assign the determined condition value to a corresponding optimization target unit. As another example, the processing circuitry 61 may display, with the display controlling function 614, the determined condition value on the display device 65, to allow the operator who has confirmed the displayed condition value to assign a given condition value that has been changed in accordance with the condition value or as necessary to the corresponding optimization target unit.

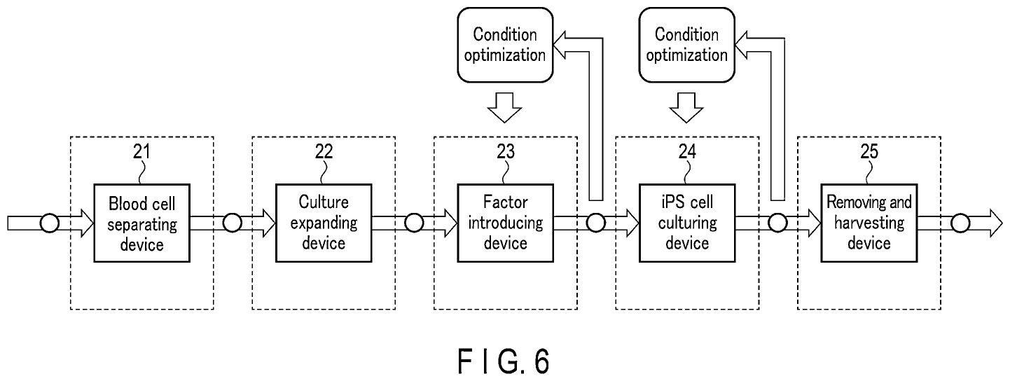

FIG. 6 schematically shows optimization of the processing condition for the first optimization target unit (the factor introducing device 23) based on the results of assessment of the first output and optimization of the processing condition for the second optimization target unit (the iPS cell culturing device 24) based on the results of assessment of the second output. As described above, by assessing the first output, determining appropriateness of the processing condition for the factor introducing device 23 based on the results of assessment of the first output, and updating the processing condition based on the results of determination, it is possible to optimize the processing condition. From another point of view, it is also possible, by assessing the first output, to verify the technical feasibility of automation of the factor introducing step. Similarly, it is also possible, by assessing the second output, to optimize the processing condition for the iPS cell culturing device 24, and to verify the technical feasibility of automation of the iPS cell culturing step.

After step S7, the optimization process ends.

The procedure for the optimization process shown in FIG. 4 is merely an example, and may be modified by deleting, adding, and/or changing one or more elements as long as such a modification does not deviate from the gist of the invention.

In the embodiment shown in FIG. 4, it has been assumed, as an example, that only the first output from the factor introducing device 23 and the second output from the iPS cell culturing device 24 are assessed; however, the configuration of the present embodiment is not limited thereto. That is, in place of the first and second outputs, a first input to the factor introducing device 23 and a second input to the iPS cell culturing device 24 may be assessed, or the first and second inputs may be assessed together with the first and second outputs.

In the embodiment shown in FIG. 4, it has been assumed, as another example, that the number of optimization target units is two (i.e., the factor introducing device 23 and the iPS cell culturing device 24); however, the configuration of the present embodiment is not limited thereto. As an example, a combination of any two of the blood cell separating device 21, the culture expanding device 22, the factor introducing device 23, the iPS cell culturing device 24, and the removing and harvesting device 25 may be adopted. The two optimization target units need not be two consecutive units in the procedure. In addition, the number of optimization target units may be one or three or more. Typically, it is desirable that at least the factor introducing device 23 and the iPS cell culturing device 24 be selected as optimization target units, as shown in the embodiment of FIG. 4, and some or all of the blood cell separating device 21, the culture expanding device 22, and the removing and harvesting device 25 may be selected as further optimization target units in addition to the factor introducing device 23 and the iPS cell culturing device 24.

In the embodiment shown in FIG. 4, it has been assumed, as another example, that each of the blood cell separating step by the blood cell separating device 21, the culture expanding step by the culture expanding device 22, the factor introducing step by the factor introducing device 23, the iPS cell culturing step by the iPS cell culturing device 24, and the removing and harvesting step by the removing and harvesting device 25 are assessed independently. However, the configuration of the present embodiment is not limited thereto. For example, if some or all of the consecutive processing steps to be performed by the blood cell separating device 21, the culture expanding device 22, the factor introducing device 23, the iPS cell culturing device 24, and the removing and harvesting device 25 for consecutive processing steps are linked, the assessing apparatus 6 may assess an input to and/or an output from some or all of the linked processing steps. It is thereby possible to optimize the processing conditions for the consecutive processing steps.

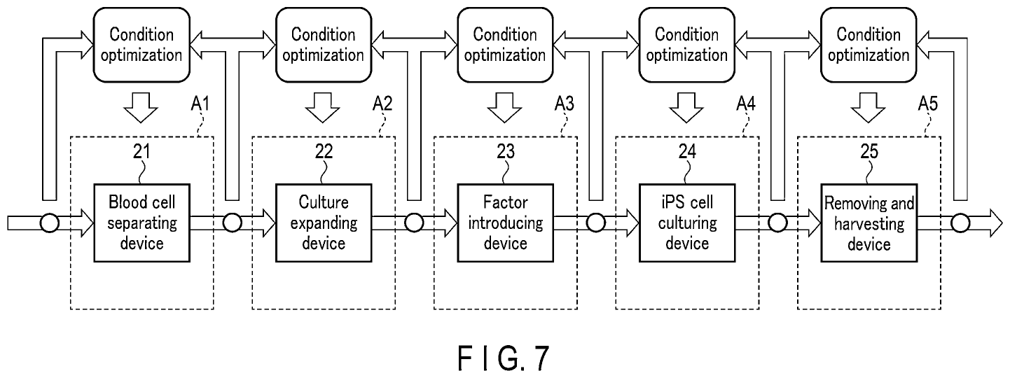

FIG. 7 is a diagram schematically showing optimization of a processing condition for each of independent processing steps A1 to A5. An independent processing step denotes a step in which each of the blood cell separating device 21, the culture expanding device 22, the factor introducing device 23, the iPS cell culturing device 24, and the removing and harvesting device 25 is independently automated. Specifically, an independent processing step denotes each of the blood cell separating step by the blood cell separating device 21, the culture expanding step by the culture expanding device 22, the factor introducing step by the factor introducing device 23, the iPS cell culturing step by the iPS cell culturing device 24, and the removing and harvesting step by the removing and harvesting device 25. As shown in FIG. 7, according to the present embodiment, it is possible to optimize the processing condition for each of the blood cell separating device 21, the culture expanding device 22, the factor introducing device 23, the iPS cell culturing device 24, and the removing and harvesting device 25 by independently assessing an input to and/or an output from each of the units 21, 22, 23, 24, and 25. In accordance therewith, it is possible to verify the technical feasibility of automation of each step.

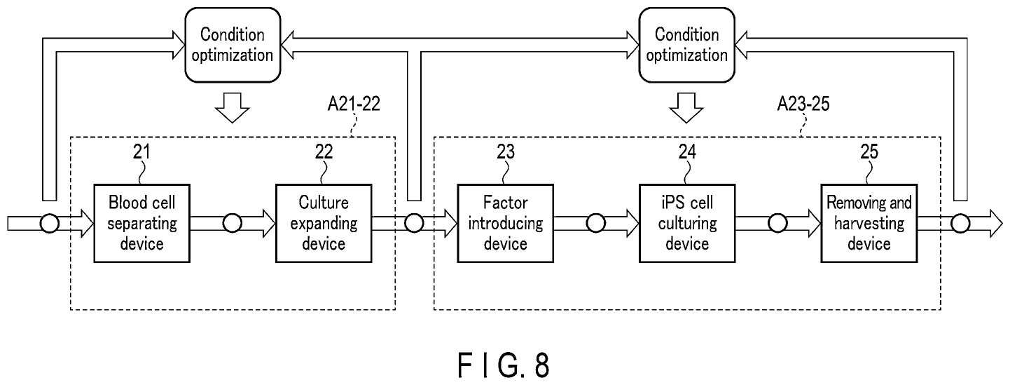

FIG. 8 is a diagram schematically showing optimization of processing conditions for partially linked steps A21-22 and A23-25. The partially linked steps A21-22 and A23-25 are processing steps formed by linking some of the consecutive processing steps. As an example, the partially linked step A21-22 denotes a processing step in which two steps, namely, the blood cell separating step by the blood cell separating device 21 and the culture expanding step by the culture expanding device 22 are linked. The partially linked step A23-25 denotes a processing step in which three steps, namely, the factor introducing step by the factor introducing device 23, the iPS cell culturing step by the iPS cell culturing device 24, and the removing and harvesting step by the removing and harvesting device 25 are linked.

It is possible to similarly assess an input to and/or an output from each of the partially linked steps A21-22 and A23-25 in accordance with the above-described optimization process. It is thereby possible to achieve optimization of processing conditions for the partially linked steps A21-22 and A23-25. An input to the blood cell separating device 21 is input to the partially linked step A21-22, and an output from the culture expanding device 22 is output from the partially linked step A21-22. An input to the factor introducing device 23 is input to the partially linked step A23-25, and an output from the removing and harvesting device 25 is output from the partially linked step A23-25. By assessing the input to and/or output from each of the partially linked steps A21-22 and A23-25, it is possible to verify the validity of automation of the partially linked step A21-22 in which the blood cell separating step and the culture expanding step are combined. Similarly, it is possible to verify the validity of automation of the partially linked step A23-25 in which the factor introducing step, the iPS cell culturing step, and the removing and harvesting step are combined.

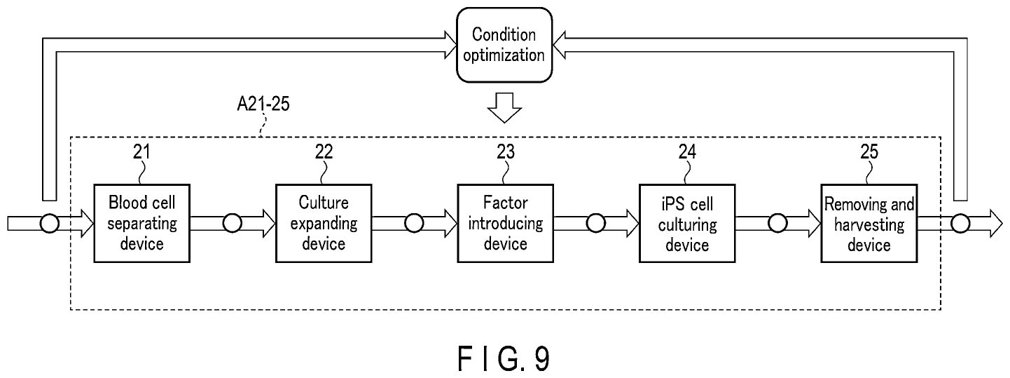

FIG. 9 is a diagram schematically showing optimization of a processing condition for a fully linked step A21-25. The fully linked step A21-25 is a processing step formed by linking all of the consecutive processing steps. Specifically, the fully linked step A21-25 denotes a processing step in which all of the blood cell separating step by the blood cell separating device 21, the culture expanding step by the culture expanding device 22, the factor introducing step by the factor introducing device 23, the iPS cell culturing step by the iPS cell culturing device 24, and the removing and harvesting step by the removing and harvesting device 25 are linked.

It is possible to similarly assess an input to and/or an output from the fully linked step A21-25 in accordance with the above-described optimization process.

It is thereby possible to achieve optimization of processing conditions for the fully linked step A21-25. An input to the factor introducing device 23 is input to the fully linked step A21-25, and an output from the removing and harvesting device 25 is output from the fully linked step A21-25. Also, by assessing the input to and/or the output from the fully linked step A21-25, it is possible to verify the validity of automation of all the constituting steps.

### (Modification 1)

In the above-described embodiment, it has been assumed that the optimization process is conducted in parallel with the iPS cell generating process by the iPS cell generating system 2. However, the configuration of the present embodiment is not limited thereto. A cell processing system 1 according to Modification 1 may conduct an optimization process independently from an iPS cell generating process by an iPS cell generating system 2. In this case, it is preferable that a switch 3q be operated so that various cell fluids flow into a measuring apparatus 4m for an input to and/or an output from an optimization target unit 2n.

### (Modification 2)

In the above-described embodiment, it has been assumed that the measuring apparatus 4 and the assessing apparatus 6 are separate. However, the configuration of the present embodiment is not limited thereto. An assessing apparatus 6 according to Modification 2 may be realized by a computer integrated into a measuring apparatus 4. If a cell processing system 1 includes a plurality of measuring apparatus 4m, as shown in FIG. 2, the assessing apparatus 6 may be integrated into some or all of the computers of each of the plurality of measuring apparatus 4m.

### (Modification 3)

In the above-described embodiment, it has been assumed that the iPS cell generating system 2 includes the blood cell separating device 21, the culture expanding device 22, the factor introducing device 23, the iPS cell culturing device 24, and the removing and harvesting device 25. However, the configuration of the present embodiment is not limited thereto. An iPS cell generating system 2 according to Modification 3 may include at least a factor introducing device 23 and an iPS cell culturing device 24. In this case, target cells extracted in advance can be input into a factor introducing device 23 via flow paths R31 and R32. Also, iPS cells cultured by the iPS cell culturing device 24 can be manually removed from a culture vessel and harvested.

### (Modification 4)

In the above-described embodiment, it has been assumed that the blood cell separating device 21, the culture expanding device 22, the factor introducing device 23, the iPS cell culturing device 24, the removing and harvesting device 25, and the measuring apparatus 4m are connected via the flow path R to allow various cell fluids to flow therethrough. However, the configuration of the present embodiment is not limited thereto. The blood cell separating device 21, the culture expanding device 22, the factor introducing device 23, the iPS cell culturing device 24, the removing and harvesting device 25, and the measuring apparatus 4m may be mechanically connected via a conveyor path for conveying containers that contain various cell fluids, or the containers may be manually conveyed.

### (General Overview)

According to some of the above-described embodiments, a cell processing system 1 includes a factor introducing device 23, an iPS cell culturing device 24, and an assessing apparatus 6. The factor introducing device 23 introduces reprogramming factors into target cells derived from a sample of a donor to develop iPS cells from the target cells. The iPS cell culturing device 24 cultures the iPS cells developed from the target cells in a culture vessel. The assessing apparatus 6 individually assesses at least an input to and/or an output from the factor introducing device 23 and an input to and/or an output from the iPS cell culturing device 24.

With the above-described configuration, since the input to and/or the output from each of the factor introducing device 23 and the iPS cell culturing device 24, each of which is automatically controlled, are individually assessed, it is possible to optimize the processing condition for the factor introducing step by the factor introducing device 23, and the processing condition for the culturing step by the iPS cell culturing device 24, while avoiding or reducing effects from the other processing steps. It is thereby possible to optimize conditions for each of the processing steps, and to generate and verify a linked processing step in which the processing steps are combined. This in turn achieves automated control of the processing steps of the entire iPS cell generating system 2, including the factor introducing device 23 and the iPS cell culturing device 24.

According to at least one of the above-described embodiments, the cell processing system 1 further includes a measuring apparatus 4 adapted to measure an input to and/or an output from the factor introducing device 23 for a first assessment item and an input to and/or an output from the iPS cell culturing device 24 for a second assessment item. The assessing apparatus 6 individually assesses the input to and/or the output from the factor introducing device 23 and the input to and/or the output from the iPS cell culturing device 24 based on a result of measurement for the first assessment item and a result of measurement for the second assessment item. Herein, the first assessment item is a ratio of CD34-positive cells, a total number of cells, and/or a ratio of living cells present in the input to the factor introducing device 23, and a total number of cells and/or a ratio of living cells present in the output from the factor introducing device 23. The second assessment item is a total number of cells and/or a ratio of living cells present in the input to the iPS cell culturing device 24, and a number, a size, and/or an intra-vessel unevenness of cell lumps present in the output from the iPS cell culturing device 24.

According to at least one of the above-described embodiments, the cell processing system 1 may further include a blood cell separating device 21 adapted to separate target cells from a sample. The assessing apparatus 6 further assesses an input to and/or an output from the blood cell separating device 21. The cell processing system 1 may further include a measuring apparatus 4 adapted to measure an input to and/or an output from the factor introducing device 23 for a first assessment item, an input to and/or an output from the iPS cell culturing device 24 for a second assessment item, and an output from the blood cell separating device 21 for a third assessment item. The assessing apparatus 6 individually assesses the input to and/or the output from the factor introducing device 23, the input to and/or the output from the iPS cell culturing device 24, and the input to and/or the output from the blood cell separating device 21 based on a result of measurement for the first assessment item, a result of measurement for the second assessment item, and a result of measurement for the third assessment item. The first assessment item is a ratio of CD34-positive cells, a total number of cells, and/or a ratio of living cells present in the input to the factor introducing device 23, and a total number of cells and/or a ratio of living cells present in the output from the factor introducing device 23. The second assessment item is a total number of cells and/or a ratio of living cells present in the input to the iPS cell culturing device 24, and a number, a size, and/or an intra-vessel unevenness of cell lumps present in the output from the iPS cell culturing device 24. The third assessment item is a number of cells present in the input to the blood cell separating device 21 and/or a presence or absence of an infectious disease in the donor, and a total number of cells and/or a ratio of living cells present in the output from the blood cell separating device 21.

With the above-described configuration, since the input to and/or the output from each of the blood cell separating device 21, the factor introducing device 23, and the iPS cell culturing device 24, each of which is automatically controlled, are individually assessed, it is possible to optimize the processing condition for the blood cell separating step by the blood cell separating device 21, the processing condition for the factor introducing step by the factor introducing device 23, and the processing condition for the culturing step by the iPS cell culturing device 24 while avoiding or reducing effects from the other processing steps. It is thereby possible to optimize conditions for each of the processing steps, and to generate and verify a linked processing step in which the processing steps are combined. This in turn achieves automated control of the processing steps of the entire iPS cell generating system 2, including the blood cell separating device 21, the factor introducing device 23, and the iPS cell culturing device 24.

According to at least one of the above-described embodiments, the cell processing system 1 may further include a culture expanding device 22 adapted to culture-expand target cells. In this case, the factor introducing device 23 introduces reprogramming factors into the culture-expanded target cells, and the assessing apparatus 6 further assesses an input to and/or an output from the culture expanding device 22. The cell processing system 1 may further include a measuring apparatus 4 adapted to measure an input to and/or an output from the factor introducing device 23 for a first assessment item, an input to and/or an output from the iPS cell culturing device 24 for a second assessment item, an input to and/or an output from the blood cell separating device 21 for a third assessment item, and an input to and/or an output from the culture expanding device 22 for a fourth assessment item. The assessing apparatus 6 individually assesses the input to and/or the output from the factor introducing device 23, the input to and/or the output from the iPS cell culturing device 24, the input to and/or the output from the blood cell separating device 21, and the input to and/or the output from the culture expanding device 22 based on a result of measurement for the first assessment item, a result of measurement for the second assessment item, a result of measurement for the third assessment item, and a result of measurement for the fourth assessment item. The first assessment item is a ratio of CD34-positive cells, a total number of cells, and/or a ratio of living cells present in the input to the factor introducing device 23, and a total number of cells and/or a ratio of living cells present in the output from the factor introducing device 23. The second assessment item is a total number of cells and/or a ratio of living cells present in the input to the iPS cell culturing device 24, and a number, a size, and/or an intra-vessel unevenness of cell lumps present in the output from the iPS cell culturing device 24. The third assessment item is a number of cells present in the input to the blood cell separating device 21 and/or a presence or absence of an infectious disease in the donor, and a total number of cells and/or a ratio of living cells present in the output from the blood cell separating device 21. The fourth assessment item is a ratio of CD34-positive cells present in the input from the culture expanding device 22, and is a ratio of CD34-positive cells, a total number of cells, and/or a ratio of living cells present in the output from the culture expanding device 22.

With the above-described configuration, since the input to and/or the output from each of the blood cell separating device 21, the culture expanding device 22, the factor introducing device 23, and the iPS cell culturing device 24, each of which is automatically controlled, are individually assessed, it is possible to optimize the processing condition for the blood cell separating step by the blood cell separating device 21, the processing condition for the culture expanding step by the culture expanding device 22, the processing condition for the factor introducing step by the factor introducing device 23, and the processing condition for the culturing step by the iPS cell culturing device 24, while avoiding or reducing effects from the other processing steps. It is thereby possible to optimize conditions for each of the processing steps, and to generate and verify a linked processing step in which the processing steps are combined. This in turn achieves automated control of the processing steps of the entire iPS cell generating system 2, including the blood cell separating device 21, the culture expanding device 22, the factor introducing device 23, and the iPS cell culturing device 24.

According to at least one of the above-described embodiments, the cell processing system 1 may further include a removing and harvesting device 25 adapted to remove cultured iPS cells from a culture vessel and harvest them. The assessing apparatus 6 further assesses an input and/or an output from the removing and harvesting device 25. The cell processing system 1 may further include a measuring apparatus 4 adapted to measure an input to and/or an output from the factor introducing device 23 for a first assessment item, an input to and/or an output from the iPS cell culturing device 24 for a second assessment item, and an input to and/or an output from the removing and harvesting device 25 for a fifth assessment item. The assessing apparatus 6 individually assesses an input to and/or an output from the factor introducing device 23, an input to and/or an output from the iPS cell culturing device 24, and an input to and/or an output from the removing and harvesting device 25 based on a result of measurement for the first assessment item, a result of measurement for the second assessment item, and a result of measurement for the fifth assessment item. The first assessment item is a ratio of CD34-positive cells, a total number of cells, and/or a ratio of living cells present in the input to the factor introducing device 23, and a total number of cells and/or a ratio of living cells present in the output from the factor introducing device 23. The second assessment item is a total number of cells and/or a ratio of living cells present in the input to the iPS cell culturing device 24, and a number, a size, and/or an intra-vessel unevenness of cell lumps present in the output from the iPS cell culturing device 24. The fifth assessment item is a number, a size, and/or an intra-vessel unevenness of cell lumps present in the input to the removing and harvesting device 25, and a total number, an undifferentiated cell marker positive rate, and a reprogramming factor retention ratio of cells present in the output from the removing and harvesting device 25.

With the above-described configuration, since the input to and/or the output from each of the factor introducing device 23 and iPS cell culturing device 24, and the removing and harvesting device 25, each of which is automatically controlled, are individually assessed, it is possible to optimize the processing condition for the factor introducing step by the factor introducing device 23, the processing condition for the culturing step by the iPS cell culturing device 24, and the processing condition for the removing and harvesting step by the removing and harvesting device 25 while avoiding or reducing effects from the other processing steps. It is thereby possible to optimize conditions for each of the processing steps, and to generate and verify a linked processing step in which the processing steps are combined. This in turn achieves automated control of the processing steps of the entire iPS cell generating system 2, including the factor introducing device 23, the iPS cell culturing device 24, and the removing and harvesting device 25.

According to at least one of the above-described embodiments, the cell processing system 1 may further include a removing and harvesting device 25 adapted to remove cultured iPS cells from a culture vessel and harvest them, and a measuring apparatus 4 adapted to measure an input to and/or an output from the factor introducing device 23 for a first assessment item, an input to and/or an output from the iPS cell culturing device 24 for a second assessment item, and an input to and/or an output from the removing and harvesting device 25 for a fifth assessment item. The measuring apparatus 4 is connected to a first branching flow path R43, which branches off from first flow paths R41 and R42 connecting the factor introducing device 23 and the iPS cell culturing device 24, and to a second branching flow path R52, which branches off from second flow paths R51 and R52 connecting the iPS cell culturing device 24 and the removing and harvesting device 25. A first switch 34 adapted to adjust a distribution of an amount of flow of an output from the factor introducing device 23 between an amount of flow to the first flow paths R41 and R42 and an amount of flow to the first branching flow path R43, and a second switch 35 adapted to adjust a distribution of an amount of flow of an output from the iPS cell culturing device 24 to the second flow paths R51 and R52 and an amount of flow to the second branching flow path R53 are further provided.

With the above-described configuration, since a flow of the inputs and/or outputs to the units 2n and/or the measuring apparatus 4 can be freely set, it is possible to perform a process of measuring and assessing the inputs and/or outputs and a process of culturing, removing, and harvesting iPS cells in parallel. It is thereby possible to reduce the burden on the operator in measuring and assessing the inputs and/or outputs, and to effectively utilize the sample, compared to the case where such processes are performed separately.

According to at least one of the above-described embodiments, the cell processing system 1 may further include a blood cell separating device 21 adapted to separate target cells from a sample, a culture expanding device 22 adapted to culture-expand target cells, a removing and harvesting device 25 adapted to remove cultured iPS cells from a culture vessel and harvest them. If some or all of the consecutive processing steps to be performed by the blood cell separating device 21, the factor introducing device 23, the iPS cell culturing device 24, and the removing and harvesting device 25 are linked, the assessing apparatus 6 assesses an input to and/or an output from some or all of the linked units. With the above-described configuration, it is possible to generate and verify a linked processing step in which the processing steps are combined. This in turn achieves automated control of the processing steps of the entire iPS cell generating system 2, including the blood cell separating device 21, the factor introducing device 23, the iPS cell culturing device 24, and the removing and harvesting device 25.

According to at least one of the above-described embodiments, the assessing apparatus 6 may maintain or change the processing condition for the factor introducing device 23 based on a result of assessment of an input to and/or an output from the factor introducing device 23, and may maintain or change the processing condition for the iPS cell culturing device 24 based on a result of assessment of an input to and/or an output from the iPS cell culturing device 24. With the above-described configuration, it is possible to realize automatic or semi-automatic optimization of conditions for the processing steps based on a result of assessment of an input and/or an output.

According to at least one of the above-described embodiments, the assessing apparatus 6 may display, on the display device 65, results of assessment of an input to and/or an output from the factor introducing device 23 and an input to and/or an output from the iPS cell culturing device 24. With the above-described configuration, it is possible for the operator to confirm the results of assessment. In addition, by confirming the results of assessment, it is possible for the operator to perform optimization of the conditions for the factor introducing device 23 and/or the iPS cell culturing device 24.

According to at least one of the above-described embodiments, it is possible to optimize the conditions for each processing step of the iPS cell generating process.

The term “processor” used in the descriptions of the embodiments means, for example, a central processing unit (CPU), a graphics processing unit (GPU), or a circuit such as an application-specific integrated circuit (ASIC), a programmable logic device (e.g., simple programmable logic device (SPLD), and a complex programmable logic device (CPLD), and a field programmable gate array (FPGA)). The processor reads and executes programs stored in storage circuitry to realize corresponding functions. The programs may be incorporated directly into circuits of the processor, instead of being stored in the storage circuitry. In this case, the processor reads the programs incorporated into the circuits and executes them to realize the functions. On the other hand, if the processor is, for example, an ASIC, the function is directly incorporated as a logic circuit into the circuit of the processor, instead of the program being stored in the storage circuit. The embodiments herein do not limit each processor to a single circuitry-type processor. Multiple independent circuits may be combined and integrated as a single processor to realize the intended functions. Furthermore, a plurality of structural components shown in any one of FIGS. 1, 2, and 3 may be integrated into a single processor to realize the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell processing system, comprising:
an introducing device configured to introduce reprogramming factors into target cells derived from a sample of a donor to develop iPS cells from the target cells;
a culturing device configured to culture the iPS cells developed from the target cells in a culture vessel; and
an assessing apparatus configured to individually assess at least an input to and/or an output from the introducing device and an input to and/or an output from the culturing device.

2. The cell processing system according to claim 1, further comprising:
a measuring apparatus configured to measure an input to and/or an output from the introducing device for a first assessment item, and an input to and/or an output from the culturing device for a second assessment item, wherein
the assessing apparatus individually assesses the input to and/or the output from the introducing device and the input to and/or the output from the culturing device based on a result of the measuring for the first assessment item and a result of the measuring for the second assessment item.

3. The cell processing system according to claim 2, wherein
the first assessment item is a ratio of CD34-positive cells, a total number of cells, and/or a ratio of living cells present in the input to the introducing device, and a total number of cells and/or a ratio of living cells present in the output from the introducing device, and
the second assessment item is a total number of cells and/or a ratio of living cells present in the input to the culturing device, and a number, a size, and/or an intra-vessel unevenness of cell lumps present in the output from the culturing device.

4. The cell processing system according to claim 1, further comprising:
a separating device configured to separate the target cells from the sample, wherein
the assessing apparatus further assesses an input to and/or an output from the separating device.

5. The cell processing system according to claim 4, further comprising:
a measuring apparatus configured to measure the input to and/or the output from the introducing device for a first assessment item, the input to and/or the output from the culturing device for a second assessment item, and the output from the separating device for a third assessment item, wherein
the assessing apparatus individually assesses the input to and/or the output from the introducing device, the input to and/or the output from the culturing device, and the input to and/or the output from the separating device based on a result of the measuring for the first assessment item, a result of the measuring for the second assessment item, and a result of the measuring for the third assessment item.

6. The cell processing system according to claim 5, wherein
the first assessment item is a ratio of CD34-positive cells, a total number of cells, and/or a ratio of living cells present in the input to the introducing device, and a total number of cells and/or a ratio of living cells present in the output from the introducing device,
the second assessment item is a total number of cells and/or a ratio of living cells present in the input to the culturing device, and a number, a size, and/or an intra-vessel unevenness of cell lumps present in the output from the culturing device, and
the third assessment item is a number of cells present in the input to the separating device and/or a presence or absence of an infectious disease in the donor, and a total number of cells and/or a ratio of living cells present in the output from the separating device.

7. The cell processing system according to claim 4, further comprising:
a culture expanding device configured to culture-expand the target cells, wherein
the introducing device introduces the reprogramming factors into the culture-expanded target cells, and
the assessing apparatus further assesses an input to and/or an output from the culture expanding device.

8. The cell processing system according to claim 7, further comprising:
a measuring apparatus configured to measure the input to and/or the output from the introducing unit for a first assessment item, the input to and/or the output from the culturing device for a second assessment item, the input to and/or the output from the separating device for a third assessment item, and the input to and/or the output from the culture expanding device for a fourth assessment, wherein
the assessing apparatus individually assesses the input to and/or the output from the introducing device, the input to and/or the output from the culturing device, the input to and/or the output from the separating device, and the input and/or output from the culture expanding device based on a result of the measuring for the first assessment item, a result of the measuring for the second assessment item, a result of the measuring for the third assessment item, and a result of the measuring for the fourth assessment item.

9. The cell processing system according to claim 8, wherein
the first assessment item is a ratio of CD34-positive cells, a total number of cells, and/or a ratio of living cells present in the input to the introducing device, and a total number of cells and/or a ratio of living cells present in the output from the introducing device,
the second assessment item is a total number of cells and/or a ratio of living cells present in the input to the culturing device, and a number, a size, and/or an intra-vessel unevenness of cell lumps present in the output from the culturing device,
the third assessment item is a number of cells present in the input to the separating device and/or a presence or absence of an infectious disease in the donor, and a total number of cells and/or a ratio of living cells present in the output from the separating device, and
the fourth assessment item is a ratio of CD34-positive cells present in the input from the culture expanding device, and is a ratio of CD34-positive cells, a total number of cells, and/or a ratio of living cells present in the output from the culture expanding device.

10. The cell processing system according to claim 1, further comprising:
a removing and harvesting device configured to remove the cultured iPS cells from the culture vessel and harvest the removed iPS cells, wherein
the assessing apparatus further assesses an input to and/or an output from the removing and harvesting device.

11. The cell processing system according to claim 10, further comprising:
a measuring apparatus configured to measure the input to and/or the output from the introducing device for a first assessment item, the input to and/or the output from the culturing device for a second assessment item, and the input to and/or the output from the removing and harvesting device for a fifth assessment item, wherein
the assessing apparatus individually assesses the input to and/or the output from the introducing device, the input to and/or the output from the culturing device, and the input to and/or the output from the removing and harvesting device based on a result of the measuring for the first assessment item, a result of the measuring for the second assessment item, and a result of the measuring for the fifth assessment item.

12. The cell processing system according to claim 11, wherein
the first assessment item is a ratio of CD34-positive cells, a total number of cells, and/or a ratio of living cells present in the input to the introducing device, and a total number of cells and/or a ratio of living cells present in the output from the introducing device,
the second assessment item is a total number of cells and/or a ratio of living cells present in the input to the culturing device, and a number, a size, and/or an intra-vessel unevenness of cell lumps present in the output from the culturing device, and
the fifth assessment item is a number, a size, and/or an intra-vessel unevenness of cell lumps present in the input to the removing and harvesting device, and a total number, an undifferentiated cell marker positive rate, and a reprogramming factor retention rate of cells present in the output from the removing and harvesting device.

13. The cell processing system according to claim 1, further comprising:
a removing and harvesting device configured to remove the cultured iPS cells from the culture vessel and harvest the removed iPS cells; and
a measuring apparatus configured to measure the input to and/or the output from the introducing unit for a first assessment item, the input to and/or the output from the culturing unit for a second assessment item, and an output from the removing and harvesting device for a fifth assessment item, wherein
the measuring apparatus is connected to a first branching flow path branching off from a first flow path connecting the introducing device and the culturing device, and to a second branching flow path branching off from a second flow path connecting the culturing device and the removing and harvesting device.

14. The cell processing system according to claim 13, further comprising:
a first switch configured to adjust a distribution of an amount of flow of the output from the introducing device between an amount of flow to the first flow path and an amount of flow to the first branching flow path; and
a second switch configured to adjust a distribution of an amount of flow of the output from the culturing device between an amount of flow to the second flow path and an amount of flow to the second branching flow path.

15. The cell processing system according to claim 1, further comprising:
a separating device configured to separate the target cells from the sample;
a culture expanding device configured to culture-expand the target cells; and
a removing and harvesting device configured to remove the cultured iPS cells from the culture vessel and harvest the removed iPS cells, wherein
if some or all of consecutive steps to be performed by the separating device, the introducing device, the culturing device, and the removing and harvesting device are linked, the assessing apparatus assesses an input to and/or an output from the linked steps.

16. The cell processing system according to claim 1, wherein
the assessing apparatus maintains or changes a processing condition for the introducing device based on a result of the assessing of the input to and/or the output from the introducing device, and maintains or changes a processing condition for the culturing device based on a result of the assessing of the input to and/or the output from the culturing device.

17. The cell processing system according to claim 1, wherein
the sample is human blood, and
the target cells are CD34-positive cells.

18. The cell processing system according to claim 1, wherein
the assessing apparatus displays, on a display device, results of the assessing of the input to and/or the output from the introducing device and the input to and/or the output from the culturing device.
